(19) United States
(12) Patent Application Publication
(10) Pub. No.: US 2008/0027373 A1
(43) Pub. Date: Jan. 31, 2008

(54) METHOD AND APPARATUS FOR THE MEASUREMENT OF INTRA-ABDOMINAL PRESSURE UTILIZING A PRESSURE TRANSDUCER

(76) Inventor: Bo Holte, Charlottenlund (DK)

Correspondence Address: ANDRUS, SCEALES, STARKE & SAWALL, LLP, 100 EAST WISCONSIN AVENUE, SUITE 1100, MILWAUKEE, WI, 53202, US

(21) Appl. No.: 11/460,509

(22) Filed: Jul. 27, 2006

(51) Int. Cl. A61M 1/00

(52) U.S. Cl. 604/27

(57) ABSTRACT

A method and apparatus for measuring the intra-abdominal pressure within a patient. The system receives a supply of discharged fluid from the patient in a drainage conduit and directs the fluid to a collection container. An air-filled sensing conduit is in fluid communication with the drainage conduit and includes a pressure transducer that detects the pressure of air within the sensing conduit. During the measurement of the intra-abdominal pressure of the patient, a measurement volume of drained fluid is returned to the patient. As the measurement volume of fluid is returned to the patient, the pressure transducer senses the pressure within the sensing conduit, which is directly related to the intra-abdominal pressure of the patient. The pressure signal from the pressure transducer is displayed either on a patient monitor or an intra-abdominal pressure monitor.

### FIELD OF THE INVENTION

The present invention relates to an apparatus and method that are useful in the measurement of the pressure within the human body abdominal cavity or a hollow viscous organ contained in the abdominal cavity. More specifically, the present invention relates to an apparatus and method that utilizes the patient's own bodily fluid collected within a collection container to measure the intra-abdominal pressure.

### BACKGROUND OF THE INVENTION

The measurement of intra-abdominal (or intra-visceral) pressure is routinely made in the clinical management of critically ill patients, or patients undergoing major surgery. Typically, the urinary bladder is the preferred site for the pressure measurement, but other hollow organs, such as the stomach, may be used as well.

Urinary bladder pressure is presently measured by connecting a pressure recording device to the patient's urinary (Foley) catheter, which empties the bladder into a drainage container. Under the prior art method, the drainage container tube is clamped and 50 cc of sterile saline is infused into the bladder from a syringe by a needle inserted into the catheter's sampling port. After infusion, the pressure in the bladder is recorded using the level of the pubic bone (symphysis pubis) as the zero pressure reference. This method is both time-consuming and presents a risk for bladder infection because the closed sterile tubing may be contaminated by the introduction of the needle when inserting the supply of saline.

The Skovlund U.S. Pat. No. 6,503,208 discloses a method and apparatus that returns a volume of collected urine from the patient back to the patient's bladder to determine the intra-abdominal pressure for the patient. The system includes a tube having a series of markings that allow a clinician to obtain a manual measurement of the intra-abdominal pressure of the patient. Although the method and system of the '208 patent provide an accurate measurement of the intra-abdominal pressure for the patient, the system requires a manual measurement to be taken by an attending clinician. Further, once the measurement has been taken, the measurement must be manually entered into an electronic database or monitoring system. Thus, a need exists for a system for monitoring the intra-abdominal pressure of the patient and displaying the sensed pressure on an automated basis.

### SUMMARY OF THE INVENTION

The present invention relates to a method and system for determining the intra-abdominal pressure of a patient. Specifically, the present invention relates to a system and method that utilizes a collected volume of fluid from the patient and returns a measurement volume of the collected fluid to the patient to determine the intra-abdominal pressure within the patient. In the first embodiment of the invention, the system includes a drainage conduit that receives discharged fluid from the patient and directs the discharged fluid to a collection container. During collection of the discharged fluid from the patient, a clamping device is closed and prevents the fluid from contaminating the filter surface.

The discharge conduit of the first embodiment of the invention includes a sensing conduit that is joined to the discharge conduit between a connection to a catheter of the patient and the collection container. In the first embodiment, the discharge conduit includes a connector that receives a first end of the sensing conduit. The second end of the sensing conduit is configured to receive a pressure transducer. The pressure transducer is preferably coupled to a patient monitor and relays pressure signals to the patient monitor related to the air pressure within the sensing conduit.

Once the pressure transducer has been connected to the second end of the sensing conduit, an intra-abdominal pressure measurement for the patient can be obtained by initially elevating the discharge conduit above the mid-axillary line of the patient until the connector between the sensing conduit and the drainage conduit is positioned at the patient's mid-axillary line. Once the discharge conduit has been properly elevated, the clamping device is released to vent the discharge conduit to atmosphere through an air filter, which maintains a sterile barrier between the fluid and the outside environment.

Once the discharge conduit has been elevated and vented, the stored supply of the patient's own fluid flows back through the drainage conduit and the catheter into a hollow organ of the patient, such as the bladder. The drainage conduit is configured such that the drainage conduit stores a sufficient volume of fluid to return the measurement volume to the patient while providing enough additional fluid to create a column of liquid above the patient's mid-axillary line that is directly related to the intra-abdominal pressure of the patient.

When the measuring volume of fluid is returned to the patient, a small amount of the fluid enters into the sensing conduit and compresses the air within the sensing conduit. The air pressure within the sensing conduit is sensed by the pressure transducer. The pressure sensed by the pressure transducer is relayed to the patient monitor, which displays the sensed intra-abdominal pressure.

In a second embodiment of the invention, a pressure monitor is utilized that incorporates an external clamping device, an internal display, an internal pressure transducer and an air dosing pump. When a pressure measurement is to be made for the patient, the pressure monitor initially clamps the drainage conduit to prevent the flow of fluid past the clamping device. Once the drainage conduit has been clamped, the pressure monitor operates the air dosing pump to inject a volume of air into the drainage conduit through a sensing conduit sufficient to return the measurement volume of fluid back to the patient's hollow organ, such as the bladder. Specifically, the air dosing pump operates to inject air into the drainage conduit until the air-to-fluid junction within the drainage conduit is positioned at the mid-axillary line of the patient. When the air-to-fluid junction is positioned at the mid-axillary line for the patient, the pressure within the sensing conduit directly represents the intra-abdominal pressure for the patient.

Various other features, objects and advantages of the invention will be made apparent from the following description taken together with the drawings.

### BRIEF DESCRIPTION OF THE DRAWINGS

The drawings illustrate the best mode presently contemplated of carrying out the invention. In the drawings:

FIG. 1 is an illustration of a prior art system for determining the intra-abdominal pressure of a patient utilizing a collected volume of fluid from the patient;

FIG. 2 is an illustration of the prior art system shown in FIG. 1 illustrating the manual measurement of the IAP of the patient utilizing markings on a manometer tube;

FIG. 3 is an illustration of the intra-abdominal pressure measuring device of the present invention as attached to a patient monitoring device having a display;

FIG. 4 is an illustration of the pressure measuring device in a position ready to determine the intra-abdominal pressure of the patient;

FIG. 5 is an illustration similar to FIG. 4 illustrating the venting of the drainage conduit to atmosphere and the determination of the intra-abdominal pressure; and

FIG. 6 is a second, alternate configuration of the intra-abdominal pressure measuring device in accordance with the present invention.

### DETAILED DESCRIPTION OF THE INVENTION

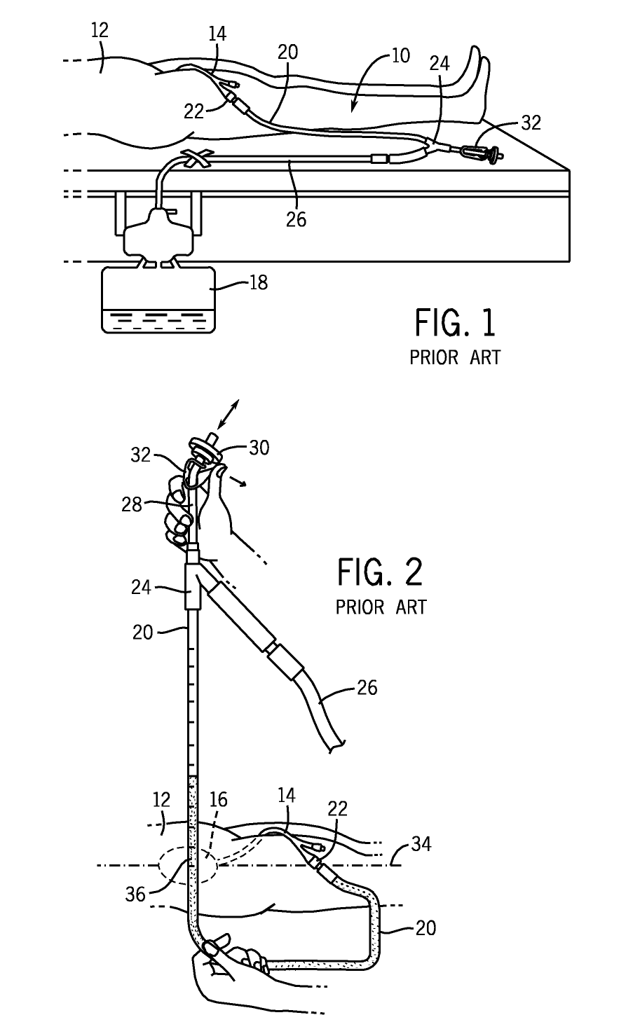

FIG. 1 illustrates a prior art intra-abdominal pressure (IAP) measuring apparatus as being used on a patient 12 in a post-operative or intensive care environment. The IAP measuring apparatus 10 shown in FIGS. 1 and 2 generally represents the device shown and described in U.S. Pat. No. 6,503,208. As illustrated in FIG. 1, the patient is in a supine position with a catheter 14, such as a urinary (Foley) catheter positioned to drain the patient's bladder 16 into a collection container 18. In the apparatus shown in FIGS. 1 and 2, a drainage conduit 20 is connected to one end 22 of the catheter 14 and provides a drainage path to the collection container 18. In the embodiment shown in FIGS. 1 and 2, the drainage conduit 20 is received within a Y-connector 24. The Y-connector 24 provides a fluid path to a second drainage conduit 26 that eventually leads to the collection container 18, as best shown in FIG. 1.

Referring now to FIG. 2, the Y-connector 24 includes a venting conduit 28 having a sterile air filter 30 and a clamping device 32. During normal operation, the clamping device 32 is in a closed position to prevent the patient's urine from coming into contact with the filter and forming deposits that may block the air filter 30 from its venting function. When the clamping device 32 is in the closed position, fluid drained from the patient 12 passes through the drainage conduit 20, the Y-connector 24 and the second drainage conduit 26 and is collected within the collection container 18.

In the embodiment shown in FIG. 2, the discharge conduit 20 is a manometer tube that includes a series of measurement markings that are calibrated in either cm H₂O or mmHg. When it is desired to make a pressure measurement for the intra-abdominal pressure for the patient 12, the Y-connector 24 is elevated above the mid-axillary line 34 of the patient 12 until the zero pressure marking line 36 is even with the mid-axillary line 34. Once the drainage conduit 20 is properly elevated, the clamping device 32 is opened, thus venting the entire drainage conduit to atmosphere. Once the drainage conduit 20 is vented to atmosphere, a measuring volume of the drained fluid from the patient returns to the patient through the catheter 14.

Once the fluid returns to the patient, the level of the fluid remaining within the discharge conduit 20 above the zero marking 36 indicates the pressure within the bladder of the patient. Since the specific gravity of the collected fluid, and specifically urine, is very close to 1.0, the bladder pressure is equivalent to the height of the urine contained within the discharge tube 20 above the zero marking 36.

Although the apparatus shown in FIGS. 1 and 2 has proven effective to determine the intra-abdominal pressure of a patient, the measurement process requires a manual viewing of the level of fluid within the discharge conduit 20 and a subsequent recording of this pressure within an automated system within the treatment environment.

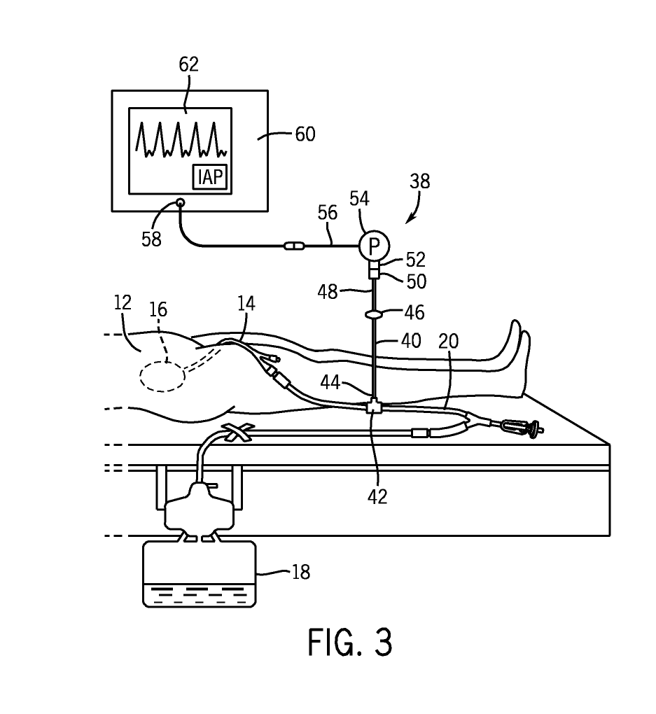

FIGS. 3-5 illustrate a first preferred embodiment of the IAP measuring apparatus 38 of the present invention, where the same reference numerals for similar components to those shown in the prior art system of FIGS. 1-2 are utilized to facilitate understanding of the present invention. As illustrated in FIG. 3, the apparatus 38 includes the drainage conduit 20 connected to the catheter 14 such that fluid from the patient 12, and specifically urine from the bladder 16, can be collected within the collection container 18. In the embodiment of the invention shown in FIG. 3, a sensing conduit 40 is positioned in fluid communication with the drainage conduit 20 through a T-shaped connector 42. The connector 42 is positioned within the drainage conduit 20 and receives a first end 44 of the sensing conduit 40. Specifically, the first end 44 of the hollow, tubular sensing conduit 40 is connected to and in fluid communication with the T-shaped connector 42. In the embodiment illustrated in FIG. 3, the sensing conduit 40 includes an air filter 46 positioned between the first end 44 and a second end 48. The second end 48 includes a coupling 50 that receives a mating coupling 52 of a pressure transducer 54. The pressure transducer 54 is a conventional component, such as the TrueWave disposable pressure transducer available from Edwards Lifesciences, and generates a signal along an output line 56 that relates to the air pressure sensed by the pressure transducer 54. The output line 56 can be received by an input 58 of a patient monitor 60 such that the patient monitor can display the pressure measurements from the transducer 54 on a display 62 on an intermittent basis when the drainage conduit is elevated above the patient and the clamping device 32 is opened to allow venting to atmospheric pressure.

As can be understood in FIG. 3, when the pressure transducer 54 is attached to the second end 48 of the sensing conduit 40, a volume of air is entrapped within the sensing conduit 40. The fluid within the discharge conduit 20 can flow into the sensing conduit 40 and compress the air therein. The trapped volume of air within the sensing conduit 40 allows the pressure transducer 54 to make a pressure determination of the fluid in the discharge conduit 20 that is determined by the air-to-liquid junction within the sensing conduit 40.

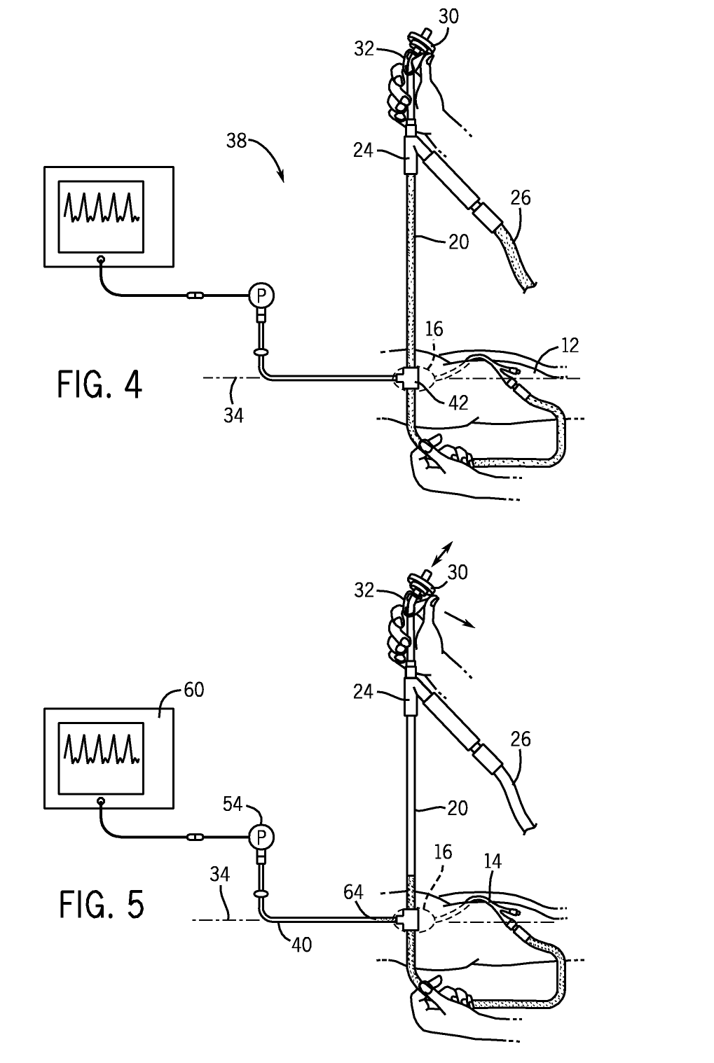

Referring now to FIG. 4, when an intra-abdominal pressure measurement is to be made utilizing the IAP measuring apparatus 38, the Y-connector 24 is elevated until the T-connector 42 is generally aligned with the mid-axillary line 34 for the patient 12. As described previously, the mid-axillary line for the patient 12 is generally level with a horizontal axis extending through the pubic bone of the patient 12 when the patient is in a supine position. As illustrated in FIG. 4, when the Y-connector 24 is initially elevated and the clamping device 32 remains closed, both the drainage conduit 20 and second drainage conduit 26 are filled with fluid collected from the patient.

Once the drainage conduit 20 and Y-connector 24 are properly elevated, the clamping device 32 is opened, causing the drainage conduit 20 to be vented to atmospheric pressure through the air filter 30. Once the drainage conduit 20 has been vented to atmosphere, a measurement volume of fluid contained within the drainage conduit 20 flows back into the patient bladder 16 through the catheter 14. Since the specific gravity of urine is very close to 1.0, the bladder pressure is equivalent to the height of the fluid contained within the discharge tube 20 provided that the liquid-to-air junction in the sensing conduit is positioned at the mid-axillary line.

As the measurement volume of the patient fluid returns to the bladder 16, a small volume of the fluid enters into the sensing conduit 40. As the fluid enters into the sensing conduit 40, the fluid meets the volume of air at a liquid-to-air junction point 64 (FIG. 5). When the volume of fluid enters the sensing conduit 40, the fluid compresses the air trapped between the pressure transducer 54 and the point 64, which is the air-to-fluid junction within the sensing conduit 40. The pressure transducer 54 senses the pressure of air within the sensing conduit 40 and generates a signal based upon the sensed pressure.

The IAP for a patient has been demonstrated to be identical to the intra-gastric pressure when the patient is supine and the patient's mid-axillary line is used as the zero pressure reference level. The greater the IAP for the patient, the further the fluid solution will travel up the sensing conduit 40. The compression of the air within the sensing conduit 40 will thus be sensed by the pressure transducer 54, which provides a signal to the patient monitor 60 that is related to the IAP for the patient 12.

In the embodiment of the invention shown in FIGS. 4 and 5, the volume of the drainage conduit 20 is sufficient to return the required measuring volume of fluid back to the patient's bladder 16 and still provide a column of fluid within the drainage conduit 20. The specific volume of the drainage conduit 20 can vary as long as the volume is sufficient to return the measurement volume to the patient during the measurement procedure.

As can be understood in FIGS. 4 and 5, once the pressure measurement has been taken, the patient monitor 60 can display the measurement as well as transmit the measurement to various other locations within a healthcare facility. The electronic registration and documentation of the IAP for the patient utilizing the system 38 of the present invention is a significant improvement over the manual measurement techniques shown in the prior art drawings of FIGS. 1 and 2.

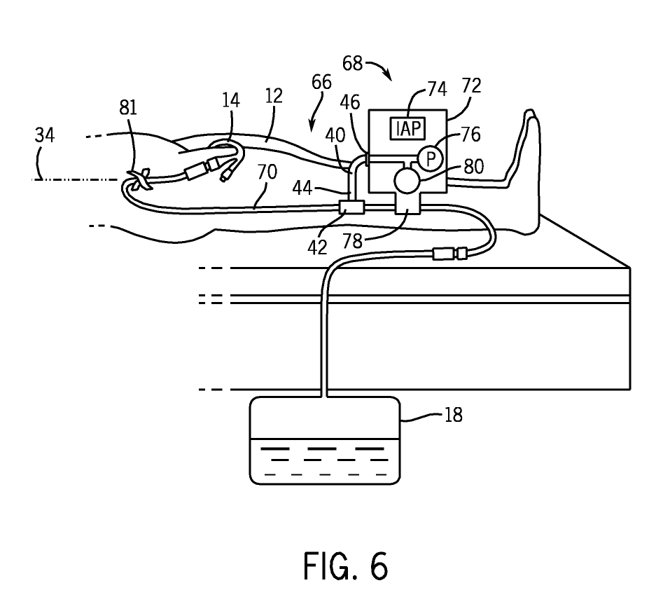

Referring now to FIG. 6, thereshown is a second embodiment of the IAP measuring apparatus, as referred to by reference numeral 66. In the second embodiment shown in FIG. 6, the apparatus includes a pressure monitor 68 that is operable to determine and display the intra-abdominal pressure for the patient 12.

As illustrated in FIG. 6, the patient 12 includes the catheter 14 connected to a single drainage conduit 70. The drainage conduit 70 drains fluid from the patient into the collection container 18 as in the first embodiment previously described.

The pressure monitor 68 includes a main housing 72 that includes a display 74. The pressure monitor is configured to connect with the air filter 46 at the second end of the sensing conduit 40. The first end 44 of the sensing conduit 40 is received by the T-connector 42 and is thus in fluid communication with the drainage conduit 70. Preferably, the air filter 46 is part of a disposable assembly including the sensing conduit 40 and has a connector that mates with the pressure monitor 68.

As illustrated in FIG. 6, the pressure monitor 68 includes an internal pressure transducer 76 that is in fluid communication through an internal passageway with the sensing conduit 40, via the air filter 46. In this manner, the pressure transducer 76 can determine the air pressure within the sensing conduit 40 in a similar manner as previously described.

The pressure monitor 68 further includes a clamping device 78 that can be selectively operated to occlude the flow of fluid through the drainage conduit 70. Specifically, the clamping device 78 is an automated device that can be operated between a first position and a second position to selectively allow fluid to flow through the drainage conduit 70.

The pressure monitor 68 includes an air dosing pump 80 that is also in fluid communication with the sensing conduit 40 through an internal passageway within the pressure monitor 68.

When the pressure monitor 68 desires to make a pressure measurement, the pressure monitor 68 initially activates the clamping device 78 to occlude the drainage conduit 70. Once the clamping device 78 has been actuated, the air pump 80 will inject a pre-set volume of air into the drainage conduit through the filter 46 and the sensing conduit 40. Since the clamping device 78 occludes the drainage conduit, the pre-set volume of air injected into the drainage conduit 70 will fill the drainage conduit and force a volume of the drained fluid collected in the drainage conduit 70 back into the bladder. Specifically, the air pump 80 will inject a volume of air sufficient to cause the fluid-to-air junction in the conduit 70 to be at the mid-axillary line 34. As illustrated, the drainage conduit 70 is held in place by a pair of adhesive strips 81 at the mid-axillary line 34. In accordance with the second embodiment shown in FIG. 6, the pressure transducer 76 can obtain a pressure measurement related to the IAP for the patient, which then can be displayed on the integral display 74.

As can be understood by the above description, the method and apparatus of the present invention utilize the pressure within the discharge conduit upon the return of a volume of fluid to a patient to determine the IAP for the patient 12. The pressure transducer senses the air pressure within a sensing conduit, which is directly related to the IAP for the patient. The pressure transducer provides a pressure signal to the patient monitor 60 or display 74, which can then display the IAP for the patient on an intermittent basis. Although two embodiments of the present invention have been shown and described in the Figures, it is contemplated by the inventor that various other methods and apparatus can be utilized for sensing the inter-abdominal pressure of the patient utilizing the pressure of the collective fluids from the patient.

I claim:

1. A method of determining the intra-abdominal pressure in a patient having a drainage conduit that drains fluid from the patient, the method comprising the steps of:
positioning a sensing conduit in fluid communication with the drainage conduit;
returning a measuring volume of the drained fluid to the patient;
measuring the air pressure within the sensing conduit after returning the measuring volume of the drained fluid to the patient; and
determining the intra-abdominal pressure of the patient based upon the measured air pressure within the sensing conduit.

2. The method of claim 1 further comprising the step of displaying the determined intra-abdominal pressure on a patient monitor.

3. The method of claim 2 further comprising the steps of:
positioning a pressure sensor at a second end of the sensing conduit; and
connecting the pressure sensor to the patient monitor.

4. The method of claim 3 wherein the measuring volume of the drained fluid returned to the patient is stored in the drainage conduit.

5. The method of claim 4 further comprising the steps of:
elevating the drainage conduit above the mid-axillary line of the patient to return the measuring volume of the drained fluid to the patient; and
positioning a junction point between the drained fluid and air within the sensing conduit at a mid-axillary line of the patient prior to measuring the air pressure within the sensing conduit.

6. The method of claim 5 wherein the measuring volume of the drain fluid is returned to the patient by venting the drainage conduit to atmosphere.

7. The method of claim 1 wherein the sensing conduit includes a volume of air.

8. The method of claim 1 further comprising the step of positioning the drainage conduit in fluid communication with a catheter inserted into a hollow organ of the patient such that the fluid from the hollow organ drains into the drainage conduit.

9. A system for determining the intra-abdominal pressure of a patient having a catheter for draining a fluid from the patient, comprising:
a drainage conduit in fluid communication with the catheter to receive the drained fluid from the patient;
a sensing conduit having a first end in fluid communication with the drainage conduit; and
a pressure transducer connected to a second end of the sensing conduit and operable to detect the air pressure within the sensing conduit when a measuring volume of fluid is returned to the patient.

10. The system of claim 9 wherein the pressure transducer is contained within an intra-abdominal pressure (IAP) monitor.

11. The system of claim 10 wherein the IAP monitor includes a display operable to display the determined intra-abdominal pressure.

12. The system of claim 11 wherein the IAP monitor includes a clamping device operable to occlude the draining conduit and a pump operable to inject a volume of air into the occluded drainage conduit to force the measuring volume of fluid back into the patient.

13. The system of claim 9 wherein the sensing conduit includes a volume of air when the pressure transducer is connected to the second end.

14. The system of claim 9 wherein the drainage conduit is sized to retain the measuring volume of fluid such that the measuring volume of fluid can be returned to the patient from the drainage conduit.

15. The system of claim 14 further comprising a clamping device coupled to the drainage conduit, wherein the clamping device is movable to an open position to vent the drainage conduit to atmosphere.

16. The system of claim 15 wherein the pressure transducer senses the intra-abdominal pressure of the patient when the measuring volume of fluid is returned to the patient.

17. A method of determining the intra-abdominal pressure in a patient having a drainage conduit that drains fluid from the patient into a collection container, the method comprising the steps of:
positioning a sensing conduit in fluid communication with the drainage conduit;
occluding the drainage conduit with a clamping device;
injecting a volume of air into the drainage conduit between the clamping device and the patient to force a measuring volume of the drained fluid into the patient;
measuring the air pressure within the sensing conduit after the measuring volume of drained fluid is returned to the patient; and
determining the intra-abdominal pressure of the patient based upon the measured air pressure within the sensing conduit.

18. The method of claim 17 further comprising the step of providing a pressure monitor having a pressure sensor operable to determine the air pressure in the sensing conduit and a pump operable to inject the volume of air into the drainage conduit.

19. The method of claim 18 wherein the pressure monitor includes a display, further comprising the step of displaying the determined intra-abdominal pressure on the display.

20. The method of claim 18 wherein the pressure monitor includes the clamping device automatically operable by the pressure monitor to occlude the drainage conduit prior to the injection of the volume of air into the drainage conduit.
